(19) United States
(12) Patent Application Publication
(10) Pub. No.: US 2007/0066601 A1
(43) Pub. Date: Mar. 22, 2007

(54) Combination of a serotonin reuptake inhibitor and a histamine 3 receptor antagonist, inverse agonist or partial agonist

(75) Inventors: Thomas Ivo Franciscus Herbert Cremers, Groningen (NL); Sandra Hogg Willigers, Bagsvaerd (DK)

Correspondence Address: DARBY & DARBY P.C., P. O. BOX 5257, NEW YORK, NY, 10150-5257, US

(73) Assignee: H. LUNDBECK A/S, VALBY-COPENHAGEN (DK)

(21) Appl. No.: 10/596,348

(22) PCT Filed: Dec. 14, 2004
(86) PCT No.: PCT/DK04/00862

§ 371 Date: Jul. 14, 2006

Related U.S. Application Data
(60) Provisional application No. 60/529,491, filed on Dec. 15, 2003.

(30) Foreign Application Priority Data
Dec. 15, 2003 (DK) PA 2003 01854

(51) Int. Cl. A61K 31/551; A61K 31/445; A61K 31/405; A61K 31/381; A61K 31/137

(52) U.S. Cl. 514/220; 514/317; 514/469; 514/419; 514/649; 514/438

(57) ABSTRACT

The invention relates to the use of a compound, which is a serotonin reuptake inhibitor, and another compound, which is a H3 receptor antagonist, inverse agonist or partial agonist for the preparation of a pharmaceutical composition for the treatment of depression, anxiety disorders and other affective disorders, such as generalized anxiety disorder, panic anxiety, obsessive compulsive disorder, acute stress disorder, post traumatic stress disorder and social anxiety disorder, eating disorders such as bulimia, anorexia and obesity, phobias, dysthymia, premenstrual syndrome, cognitive disorders, impulse control disorders, attention deficit hyperactivity disorder, drug abuse or any other disorder responsive to serotonin reuptake inhibitor.

The present invention relates to the combination of a serotonin reuptake inhibitor (SRI) and a histamine 3(H₃) receptor antagonist. Accordingly, the present invention relates to the use of certain compounds, and to compositions of compounds having serotonin reuptake inhibiting activity and H₃ antagonistic, partial agonistic or inverse agonistic activity for the treatment of depression and other affective disorders. The combined serotonin reuptake inhibiting effect and the H₃ antagonistic, partial agonistic or inverse agonistic effect may reside within the same chemical entity or in two separate chemical entities.

### BACKGROUND

Selective serotonin reuptake inhibitors (hereinafter referred to as SSRIs) have become first choice therapeutics in the treatment of depression, certain forms of anxiety and social phobias, because they are effective, well tolerated and have a favourable safety profile compared to the classic tricyclic antidepressants.

However, clinical studies on depression and anxiety disorders indicate that non-response to SSRIs is substantial, up to 30%. Another, often neglected, factor in antidepressant treatment is compliance, which has a rather profound effect on the patient's motivation to continue pharmacotherapy.

First of all, there is the delay in therapeutic effect of SSRIs. Sometimes symptoms even worsen during the first weeks of treatment. Secondly, sexual dysfunction is a side effect common to all SSRIs. Without addressing these problems, real progress in the pharmacotherapy of depression and anxiety disorders is not likely to happen.

In order to cope with non-response, psychiatrists sometimes make use of augmentation strategies. Augmentation of antidepressant therapy may be accomplished through the co-administration of mood stabilizers such as lithium carbonate or triiodothyronin or by the use of electroshock.

In 1993, an augmentation strategy with pindolol was described by Artigas et al. in Trends Pharmacol. Sci. 1993, 14, p 262-263. Artigas' idea is based on intracerebral microdialysis experiments in animals. In fact, later neurochemical studies built on the desensitization hypothesis by Blier and co-workers stated that the delay in therapeutic effect of antidepressants is related to a gradual desensitization of 5-HT autoreceptors (Blier et al. J. Clin. Psycipharmacol. 1987, 7 suppl. 6, 24S-35S). A key point in their hypothesis is that the effects of SSRIs on the release-controlling somatodendritic autoreceptors (5-HT_{1A}) limit the release of 5-HT in terminal areas and thus the effect of 5-HT uptake inhibition in those regions. This is supported by microdialysis experiments in rats, showing that the increase in extracellular 5-HT elicited by a single dose of an SSRI is augmented by co-administration of a 5-HT_{1A} autoreceptor antagonist (Invernizzi et al. Brain Res, 1992, 584, p 322-324 and Hjorth, S., J. Neurochem, 1993, 60, p 776-779).

The effect of combined administration of a compound that inhibits serotonin reuptake and a 5-HT_{1A} receptor antagonist has been evaluated in several studies (Innis, R. B. et al. Eur. J. Pharmacol. 1987, 143, p. 1095-204 and Gartside, S. E., Br. J. Pharmacol, 1995, 115, p 1064-1070, Blier, P. et al. Trends in Pharmacol. Science 1994, 15, 220). In these studies it was found that 5-HT_{1A} receptor antagonists would abolish the initial brake on 5-HT neurotransmission induced by the serotonin reuptake inhibitors and thus produce an immediate boost of 5-HT transmission and a rapid onset of therapeutic action.

Several patent applications have been filed which cover the use of a combination of a 5-HT_{1A} antagonist and a serotonin reuptake inhibitor for the treatment of depression (see e.g. EP-A2-687 472 and EP-A2-714 663).

Another approach to increase terminal 5-HT would be through blockade of the 5-HT_{1 B} autoreceptor. Microdialysis experiments in rats have indeed shown that increase of hippocampal 5-HT by citalopram is potentiated by GMC 2-29, an experimental 5-HT_{1B} receptor antagonist.

Several patent applications covering the combination of an SSRI and a 5-HT_{1B} antagonist or partial agonist have also been filed (WO 97/28141, WO 96/03400, EP-A-701819 and WO 99/13877).

Histamine is an important neurotransmitter in the brain. It has been implicated in the regulation of numerous important activities of the central nervous system as arousal, cognition, circadian rythms and neuroendocrine regulation (Fernandez-Novoa L., Cacabelos R. Behav. Brain Res., vol 124 (2), pp. 213-233 (2001)). Histamine, noradrenaline, and serotonin are all under inhibitory control of the histmaminergic system via H3 auto- and heteroceptors (Schlicker et al. Naunyn Schiedebergs Arch Pharmacol., vol 377 (5): 588-590 (1988), DiCarlo G., Ghi P., Orsetti M. Prog. Neuro-psychopharmacol. & Biol. Psychiat., vol 24, pp 275-284 (2000). Arrang J. M., Garbarg M., Schwartz J. C., Nature, vol 302, pp 832-837 (1983).) Electrical evoked release of tritiated serotonin from cortex synaptosomes was inhibited by application of H3 agonist R-(alphe)-methylhistamine. This effect was reversed by the H3 antagonist thioperamide (Fink K., Schlicker E., Neise A., Gothert M., Naunyn Schiedebergs Arch Pharmacol., Vol. 342 (5): 513-519 (1990)).

### THE INVENTION

It has now surprisingly been found that a H₃ antagonist will augment the effect of an SRI, in particular an SSRI, on extracellular 5-HT levels.

It is therefore suggested that the combination of an SRI, in particular an SSRI, and a H₃ antagonist or a molecule, which has both 5-HT reuptake inhibitory and H₃ antagonistic properties, would have a better efficacy and faster onset than an SRI, in particular an SSRI, alone.

Antagonism at any H₃ splice variants, including possible subtypes is claimed.

This invention covers both SSRI plus H₃ antagonist in separate or the same molecule.

The present invention thus provides:

The use of a H₃ receptor antagonist, inverse agonist or partial agonist for the preparation of a pharmaceutical composition to be used in combination with a serotonin reuptake inhibitor (SRI).

The present invention relates to the use of a compound, which is a serotonin reuptake inhibitor, and another compound, which is a H₃ receptor antagonist, inverse agonist or partial agonist for the preparation of a pharmaceutical composition for the treatment of depression, anxiety disorders and other affective disorders, such as generalized anxiety disorder, panic anxiety, obsessive compulsive disorder, acute stress disorder, post traumatic stress disorder and social anxiety disorder, eating disorders such as bulimia, anorexia and obesity, phobias, dysthymia, premenstrual syndrome, cognitive disorders, impulse control disorders, attention deficit hyperactivity disorder, drug abuse or any other disorder responsive to serotonin reuptake inhibitors.

The present invention also relates to the use of a H₃ receptor antagonist, inverse agonist or partial agonist for the preparation of a pharmaceutical composition useful for augmenting and/or providing faster onset of the therapeutic effect of a serotonin reuptake inhibitor.

In a preferred embodiment, the invention relates to the use as above wherein the serotonin reuptake inhibitor is used for the treatment of depression, anxiety disorders and other affective disorders, including generalized anxiety disorder, panic anxiety, obsessive compulsive disorder, acute stress disorder, post traumatic stress disorder or social anxiety disorder, eating disorders such as bulimia, anorexia and obesity, phobias, dysthymia, premenstrual syndrome, cognitive disorders, impulse control disorders, attention deficit hyperactivity disorder, drug abuse and any other disorder responsive to a SRI.

In another embodiment, the invention relates to the use of
a) a compound which is a serotonin reuptake inhibitor and a H₃ receptor antagonist, inverse agonist or partial agonist, or
b) a combination of a compound, which is a serotonin reuptake inhibitor, and a compound, which is a H₃ receptor antagonist, inverse agonist or partial agonist,
   for the preparation of a pharmaceutical composition or kit (kit-of-parts) useful for the treatment of depression, anxiety disorders and other affective disorders, such as generalized anxiety disorder, panic anxiety, obsessive compulsive disorder, acute stress disorder, post traumatic stress disorder and social anxiety disorder, eating disorders such as bulimia, anorexia and obesity, phobias, dysthymia, premenstrual syndrome, cognitive disorders, impulse control disorders, attention deficit hyperactivity disorder, drug abuse or any other disorder responsive to serotonin reuptake inhibitors.

In two independent embodiments, the invention relates to the use of a compound, which is a serotonin reuptake inhibitor, and a compound, which is a H₃ receptor antagonist, inverse agonist or partial agonist, for the preparation of a:
(a) pharmaceutical composition, or
(b) kit
   useful for the treatment of depression, anxiety disorders and other affective disorders, such as generalized anxiety disorder, panic anxiety, obsessive compulsive disorder, acute stress disorder, post traumatic stress disorder and social anxiety disorder, eating disorders such as bulimia, anorexia and obesity, phobias, dysthymia, premenstrual syndrome, cognitive disorders, impulse control disorders, attention deficit hyperactivity disorder, drug abuse or any other disorder responsive to serotonin reuptake inhibitors.

In a further embodiment, the invention relates to a pharmaceutical composition or kit comprising:
a) a compound, which is a serotonin reuptake inhibitor, and a H₃ receptor antagonist, inverse agonist or partial agonist, or
b) a combination of a compound, which is a serotonin reuptake inhibitor, and another compound, which is a H₃ receptor antagonist, inverse agonist or partial agonist,
   and optionally pharmaceutically acceptable carriers or diluents.

In two further individual embodiments, the invention relates to either a pharmaceutical composition or a kit comprising a compound, which is a serotonin reuptake inhibitor, and another compound, which is a H₃ receptor antagonist, inverse agonist or partial agonist, and optionally pharmaceutically acceptable carriers or diluents.

In yet another embodiment, the invention relates to a method for the treatment of depression, anxiety disorders and other affective disorders, such as generalized anxiety disorder, panic anxiety, obsessive compulsive disorder, acute stress disorder, post traumatic stress disorder and social anxiety disorder, eating disorders such as bulimia, anorexia and obesity, phobias, dysthymia, premenstrual syndrome, cognitive disorders, impulse control disorders, attention deficit hyperactivity disorder, drug abuse or any other disorder responsive to serotonin reuptake inhibitors comprising administering to a person in need thereof a therapeutically effective amount of
a) a compound, which is a serotonin reuptake inhibitor, and a H₃ receptor antagonist, inverse agonist or partial agonist, or
b) a combination of a compound, which is a serotonin reuptake inhibitor and a compound, which is a H₃ receptor antagonist, inverse agonist or partial agonist.

Whenever mentioned, each of the options
a) a compound, which is a serotonin reuptake inhibitor, and a H₃ receptor antagonist, inverse agonist or partial agonist, and
b) a combination of a compound, which is a serotonin reuptake inhibitor and a (or another) compound, which is a H₃ receptor antagonist, inverse agonist or partial agonist
   are intended to be individual embodiments. Accordingly, each of them may be claimed individually.

Each of the medical indications depression, anxiety disorders and other affective disorders, including generalized anxiety disorder, panic anxiety, obsessive compulsive disorder, acute stress disorder, post traumatic stress disorder or social anxiety disorder, eating disorders such as bulimia, anorexia and obesity, phobias, dysthymia, premenstrual syndrome, cognitive disorders, impulse control disorders, attention deficit hyperactivity disorder, drug abuse and any other disorder responsive to a SRI is intended to be an individual embodiment. Accordingly, whenever mentioned in the present description, each of the indications specified above may be claimed individually.

Whenever the indications depression, anxiety disorders and other affective disorders, including generalized anxiety disorder, panic anxiety, obsessive compulsive disorder, acute stress disorder, post traumatic stress disorder or social anxiety disorder, eating disorders such as bulimia, anorexia and obesity, phobias, dysthymia, premenstrual syndrome, cognitive disorders, impulse control disorders, attention deficit hyperactivity disorder, drug abuse and any other disorder responsive to a SRI are mentioned in relation to use of a H₃ receptor antagonist, inverse agonist or partial agonist and an SRI, a pharmaceutical composition, a kit, a method of treatment and a method for the identification of compounds useful for treatment it is intended to be an individual embodiment. Accordingly, each of the indications specified above may individually be claimed together with said use of a H₃ receptor antagonist, inverse agonist or partial agonist and an SRI, pharmaceutical composition, kit, method of treatment and method for the identification of compounds useful for treatment.

In a particular embodiment, a selective serotonin reuptake inhibitor is used according to the invention.

In another particular embodiment, a compound, which is selective for the H₃ receptor is used according to the invention.

In a further embodiment, a compound, which is an antagonist, an inverse agonist at the H₃ receptor is used according to the invention.

The pharmaceutical composition or kit according to the invention may be administered by simultaneous administration. The term “simultaneous administration” as used herein means, that the H₃ receptor antagonist, inverse agonist or partial agonist and the SRI are administered with a time separation of no more than 15 minutes, such as at most 10 minutes, such as at most 5 minutes or such as at most 2 minutes. The H₃ receptor antagonist, inverse agonist or partial agonist and the SRI may be contained in the “same unit dosage form” or in “discrete dosage forms”. As used herein, the term “same unit dosage form” means a dosage form comprising both the SRI and the H₃ receptor antagonist, inverse agonist or partial agonist. As used herein, the term “discrete dosage form” means that the H₃ receptor antagonist, inverse agonist or partial agonist is comprised in one dosage form and that the SRI is comprised in another dosage form.

Simultaneous administration of H₃ receptor antagonist, inverse agonist or partial agonist and the SRI is optionally combined with administration of supplementary doses of H₃ receptor antagonist, inverse agonist or partial agonist. The supplementary doses of H₃ receptor antagonist, inverse agonist or partial agonist may be given for instance 1, 2, 3 or 4 times a day whereas the SRI and the H₃ receptor antagonist, inverse agonist or partial agonist which are administered by “simultaneous administration” may be given one or more times a day, e.g. once daily or e.g. twice daily. Accordingly:
the H₃ receptor antagonist, inverse agonist or partial agonist and the SRI may be administered by simultaneous administration once daily and supplementary doses of H₃ receptor antagonist, inverse agonist or partial agonist may be administered 1, 2, 3 or 4 times a day, such as 1, 2 or 3 times a day, such as once or twice daily, such as twice daily or such as once daily,
   or
the H₃ receptor antagonist, inverse agonist or partial agonist and the SRI may be administered by simultaneous administration twice daily and supplementary doses of H₃ receptor antagonist, inverse agonist or partial agonist may be administered 1, 2, 3 or 4 times a day, such as 1, 2 or 3 times a day, such as once or twice daily, such as twice daily or such as once daily.

Alternatively, the pharmaceutical composition or kit according to the invention is administered by sequential administration. The term “sequential administration” as used herein means that 1 or more daily doses of H₃ receptor antagonist, inverse agonist or partial agonist and 1 or more daily dose H₃s of SRI are administered with a time separation between two administered doses of more than 15 minutes and less than 4 hours, such as more than 2 hours and less than 4 hours, such as more than 15 minutes and less than 2 hours, such as more than 1 hour and less than 2 hours, such as more than 30 minutes and less than 1 hour, such as more than 15 minutes and less than 30 minutes. Either, the SRI or the H₃ receptor antagonist, inverse agonist or partial agonist may be administered first. The H₃ receptor antagonist, inverse agonist or partial agonist and the SRI are contained in discrete dosage forms, optionally contained in the same container or package. Typically, 1, 2, 3, 4 or 5 daily doses of H₃ receptor antagonist, inverse agonist or partial agonist and 1 or 2 daily doses of SRI may be administered. Accordingly:
the H₃ receptor antagonist, inverse agonist or partial agonist and the SRI may be administered once daily and the H₃ receptor antagonist, inverse agonist or partial agonist may be administered 1, 2, 3, 4 or 5 times a day, such as 1, 2, 3 or 4 times a day, such as 1, 2 or 3 times a day, such as once or twice daily, such as twice daily or such as once daily,
   or
the H₃ receptor antagonist, inverse agonist or partial agonist and the SRI may be administered twice daily and the H₃ receptor antagonist, inverse agonist or partial agonist may be administered 1, 2, 3, 4 or 5 times a day, such as 1, 2, 3 or 4 times a day, such as 1, 2 or 3 times a day, such as once or twice daily, such as twice daily or such as once daily.

Accordingly, the pharmaceutical composition or kit according to the invention may be adapted for simultaneous administration of the active ingredients, or it may be adapted for sequential administration of the active ingredients. When the pharmaceutical composition or kit is adapted for simultaneous administration, the active ingredients may be contained in the same unit dosage form. When the pharmaceutical composition or kit is adapted for sequential administration, the active ingredients are contained in discrete dosage forms, optionally contained in the same container or package. As used herein, an “active ingredient” means an SRI or a H₃ receptor antagonist, inverse agonist or partial agonist.

A kit (kit-of-parts) comprises a preparation of the H₃ receptor antagonist, inverse agonist or partial agonist in a first-unit dosage form, and the SRI in a second-unit dosage form, and container means for containing said first and second dosage forms.

In particular, the present invention relates to the use of, and to pharmaceutical compositions or kits comprising the following combinations:

Thioperamide and an SRI selected from citalopram, escitalopram, fluoxetine, sertraline, paroxetine, fluvoxamine, venlafaxine, dapoxetine, vilazodone, duloxetine, nefazodone, imipramine, femoxetine and clomipramine.

Ciproxifan and an SRI selected from citalopram, escitalopram, fluoxetine, sertraline, paroxetine, fluvoxamine, venlafaxine, dapoxetine, duloxetine, vilazodone, nefazodone, imipramine, femoxetine and clomipramine.

Iodophenpropit and an SRI selected from citalopram, escitalopram, fluoxetine, sertraline, paroxetine, fluvoxamine, venlafaxine, dapoxetine, duloxetine, vilazodone, nefazodone, imipramine, femoxetine and clomipramine.

GR 168320 and an SRI selected from citalopram, escitalopram, fluoxetine, sertraline, paroxetine, fluvoxamine, venlafaxine, dapoxetine, duloxetine, vilazodone, nefazodone, imipramine, femoxetine and clomipramine.

GR 175737 and an SRI selected from citalopram, escitalopram, fluoxetine, sertraline, paroxetine, fluvoxamine, venlafaxine, dapoxetine, duloxetine, vilazodone, nefazodone, imipramine, femoxetine and clomipramine.

Iodoproxyfan and an SRI selected from citalopram, escitalopram, fluoxetine, sertraline, paroxetine, fluvoxamine, venlafaxine, dapoxetine, duloxetine, vilazodone, nefazodone, imipramine, femoxetine and clomipramine.

Proxifan and an SRI selected from citalopram, escitalopram, fluoxetine, sertraline, paroxetine, fluvoxamine, venlafaxine, dapoxetine, duloxetine, vilazodone, nefazodone, imipramine, femoxetine and clomipramine.

Perceptin (GT 2331) and an SRI selected from citalopram, escitalopram, fluoxetine, sertraline, paroxetine, fluvoxamine, venlafaxine, dapoxetine, duloxetine, vilazodone, nefazodone, imiparmin, femoxetine and clomipramine.

JB 98064 and an SRI selected from citalopram, escitalopram, fluoxetine, sertraline, paroxetine, fluvoxamine, venlafaxine, dapoxetine, duloxetine, vilazodone, nefazodone, imipramine, femoxetine and clomipramine.

VUF 4163 and an SRI selected from citalopram, escitalopram, fluoxetine, sertraline, paroxetine, fluvoxamine, venlafaxine, dapoxetine, duloxetine, vilazodone, nefazodone, imipramine, femoxetine and clomipramine.

VUF 5000 and an SRI selected from citalopram, escitalopram, fluoxetine, sertraline, paroxetine, fluvoxamine, venlafaxine, dapoxetine, duloxetine, vilazodone, nefazodone, imipramine, femoxetine and clomipramine.

VUF 5182 and an SRI selected from citalopram, escitalopram, fluoxetine, sertraline, paroxetine, fluvoxamine, venlafaxine, dapoxetine, duloxetine, vilazodone, nefazodone, imipramine, femoxetine and clomipramine.

VUF 9153 and an SRI selected from citalopram, escitalopram, fluoxetine, sertraline, paroxetine, fluvoxamine, venlafaxine, dapoxetine, duloxetine, vilazodone, nefazodone, imipramine, femoxetine and clomipramine.

A 923 and an SRI selected from citalopram, escitalopram, fluoxetine, sertraline, paroxetine, fluvoxamine, venlafaxine, dapoxetine, duloxetine, vilazodone, nefazodone, imipramine, femoxetine and clomipramine.

A 304121 and an SRI selected from citalopram, escitalopram, fluoxetine, sertraline, paroxetine, fluvoxamine, venlafaxine, dapoxetine, duloxetine, vilazodone, nefazodone, imipramine, femoxetine and clomipramine.

A 317920 and an SRI selected from citalopram, escitalopram, fluoxetine, sertraline, paroxetine, fluvoxamine, venlafaxine, dapoxetine, duloxetine, vilazodone, nefazodone, imipramine, femoxetine and clomipramine.

A 320436 and an SRI selected from citalopram, escitalopram, fluoxetine, sertraline, paroxetine, fluvoxamine, venlafaxine, dapoxetine, duloxetine, vilazodone, nefazodone, imipramine, femoxetine and clomipramine.

A 331440 and an SRI selected from citalopram, escitalopram, fluoxetine, sertraline, paroxetine, fluvoxamine, venlafaxine, dapoxetine, duloxetine, vilazodone, nefazodone, imipramine, femoxetine and clomipramine.

A 349413 and an SRI selected from citalopram, escitalopram, fluoxetine, sertraline, paroxetine, fluvoxamine, venlafaxine, dapoxetine, duloxetine, vilazodone, nefazodone, imipramine, femoxetine and clomipramine.

A 349821 and an SRI selected from citalopram, escitalopram, fluoxetine, sertraline, paroxetine, fluvoxamine, venlafaxine, dapoxetine, duloxetine, vilazodone, nefazodone, imipramine, femoxetine and clomipramine.

A 417022 and an SRI selected from citalopram, escitalopram, fluoxetine, sertraline, paroxetine, fluvoxamine, venlafaxine, dapoxetine, duloxetine, vilazodone, nefazodone, imipramine, femoxetine and clomipramine.

A 423579 and an SRI selected from citalopram, escitalopram, fluoxetine, sertraline, paroxetine, fluvoxamine, venlafaxine, dapoxetine, duloxetine, vilazodone, nefazodone, imipramine, femoxetine and clomipramine.

A 424835 and an SRI selected from citalopram, escitalopram, fluoxetine, sertraline, paroxetine, fluvoxamine, venlafaxine, dapoxetine, duloxetine, vilazodone, nefazodone, imipramine, femoxetine and clomipramine.

A 431404 and an SRI selected from citalopram, escitalopram, fluoxetine, sertraline, paroxetine, fluvoxamine, venlafaxine, dapoxetine, duloxetine, vilazodone, nefazodone, imipramine, femoxetine and clomipramine.

ABT 239 and an SRI selected from citalopram, escitalopram, fluoxetine, sertraline, paroxetine, fluvoxamine, venlafaxine, dapoxetine, duloxetine, vilazodone, nefazodone, imipramine, femoxetine and clomipramine.

ABT 834 and an SRI selected from citalopram, escitalopram, fluoxetine, sertraline, paroxetine, fluvoxamine, venlafaxine, dapoxetine, duloxetine, vilazodone, nefazodone, imipramine, femoxetine and clomipramine.

AQ 0145 and an SRI selected from citalopram, escitalopram, fluoxetine, sertraline, paroxetine, fluvoxamine, venlafaxine, dapoxetine, duloxetine, vilazodone, nefazodone, imipramine, femoxetine and clomipramine.

FUB 181 and an SRI selected from citalopram, escitalopram, fluoxetine, sertraline, paroxetine, fluvoxamine, venlafaxine, dapoxetine, duloxetine, vilazodone, nefazodone, imipramine, femoxetine and clomipramine.

FUB 360 and an SRI selected from citalopram, escitalopram, fluoxetine, sertraline, paroxetine, fluvoxamine, venlafaxine, dapoxetine, duloxetine, vilazodone, nefazodone, imipramine, femoxetine and clomipramine.

FUB 407 and an SRI selected from citalopram, escitalopram, fluoxetine, sertraline, paroxetine, fluvoxamine, venlafaxine, dapoxetine, duloxetine, vilazodone, nefazodone, imipramine, femoxetine and clomipramine.

FUB 637 and an SRI selected from citalopram, escitalopram, fluoxetine, sertraline, paroxetine, fluvoxamine, venlafaxine, dapoxetine, duloxetine, vilazodone, nefazodone, imipramine, femoxetine and clomipramine.

FUB 836 and an SRI selected from citalopram, escitalopram, fluoxetine, sertraline, paroxetine, fluvoxamine, venlafaxine, dapoxetine, duloxetine, vilazodone, nefazodone, imipramine, femoxetine and clomipramine.

GSK189254A and an SRI selected from citalopram, escitalopram, fluoxetine, sertraline, paroxetine, fluvoxamine, venlafaxine, dapoxetine, duloxetine, vilazodone, nefazodone, imipramine, femoxetine and clomipramine.

GSK 207040A and an SRI selected from citalopram, escitalopram, fluoxetine, sertraline, paroxetine, fluvoxamine, venlafaxine, dapoxetine, duloxetine, vilazodone, nefazodone, imipramine, femoxetine and clomipramine.

GT 2016 and an SRI selected from citalopram, escitalopram, fluoxetine, sertraline, paroxetine, fluvoxamine, venlafaxine, dapoxetine, duloxetine, vilazodone, nefazodone, imipramine, femoxetine and clomipramine.

GT 2104 and an SRI selected from citalopram, escitalopram, fluoxetine, sertraline, paroxetine, fluvoxamine, venlafaxine, dapoxetine, duloxetine, vilazodone, nefazodone, imipramine, femoxetine and clomipramine.

GT 2209 and an SRI selected from citalopram, escitalopram, fluoxetine, sertraline, paroxetine, fluvoxamine, venlafaxine, dapoxetine, duloxetine, vilazodone, nefazodone, imipramine, femoxetine and clomipramine.

GT 2212 and an SRI selected from citalopram, escitalopram, fluoxetine, sertraline, paroxetine, fluvoxamine, venlafaxine, dapoxetine, duloxetine, vilazodone, nefazodone, imipramine, femoxetine and clomipramine.

GT 2227 and an SRI selected from citalopram, escitalopram, fluoxetine, sertraline, paroxetine, fluvoxamine, venlafaxine, dapoxetine, duloxetine, vilazodone, nefazodone, imipramine, femoxetine and clomipramine.

GT 2232 and an SRI selected from citalopram, escitalopram, fluoxetine, sertraline, paroxetine, fluvoxamine, venlafaxine, dapoxetine, duloxetine, vilazodone, nefazodone, imipramine, femoxetine and clomipramine.

GT 2390 and an SRI selected from citalopram, escitalopram, fluoxetine, sertraline, paroxetine, fluvoxamine, venlafaxine, dapoxetine, duloxetine, vilazodone, nefazodone, imipramine, femoxetine and clomipramine.

GT 2349 and an SRI selected from citalopram, escitalopram, fluoxetine, sertraline, paroxetine, fluvoxamine, venlafaxine, dapoxetine, duloxetine, vilazodone, nefazodone, imipramine, femoxetine and clomipramine.

GT 2355 and an SRI selected from citalopram, escitalopram, fluoxetine, sertraline, paroxetine, fluvoxamine, venlafaxine, dapoxetine, duloxetine, vilazodone, nefazodone, imipramine, femoxetine and clomipramine.

GT 2394 and an SRI selected from citalopram, escitalopram, fluoxetine, sertraline, paroxetine, fluvoxamine, venlafaxine, dapoxetine, duloxetine, vilazodone, nefazodone, imipramine, femoxetine and clomipramine.

Imoproxifan and an SRI selected from citalopram, escitalopram, fluoxetine, sertraline, paroxetine, fluvoxamine, venlafaxine, dapoxetine, duloxetine, vilazodone, nefazodone, imipramine, femoxetine and clomipramine.

Impentamine and an SRI selected from citalopram, escitalopram, fluoxetine, sertraline, paroxetine, fluvoxamine, venlafaxine, dapoxetine, duloxetine, vilazodone, nefazodone, imipramine, femoxetine and clomipramine.

JNJ 5207852 and an SRI selected from citalopram, escitalopram, fluoxetine, sertraline, paroxetine, fluvoxamine, venlafaxine, dapoxetine, duloxetine, vilazodone, nefazodone, imipramine, femoxetine and clomipramine.

NNC 0038 0000 1049 and an SRI selected from citalopram, escitalopram, fluoxetine, sertraline, paroxetine, fluvoxamine, venlafaxine, dapoxetine, duloxetine, vilazodone, nefazodone, imipramine, femoxetine and clomipramine.

NNC 0038 0000 1202 and an SRI selected from citalopram, escitalopram, fluoxetine, sertraline, paroxetine, fluvoxamine, venlafaxine, dapoxetine, duloxetine, vilazodone, nefazodone, imipramine, femoxetine and clomipramine.

SCH 50971 and an SRI selected from citalopram, escitalopram, fluoxetine, sertraline, paroxetine, fluvoxamine, venlafaxine, dapoxetine, duloxetine, vilazodone, nefazodone, imipramine, femoxetine and clomipramine.

SCH 79687 and an SRI selected from citalopram, escitalopram, fluoxetine, sertraline, paroxetine, fluvoxamine, venlafaxine, dapoxetine, duloxetine, vilazodone, nefazodone, imipramine, femoxetine and clomipramine.

UCL 1199 and an SRI selected from citalopram, escitalopram, fluoxetine, sertraline, paroxetine, fluvoxamine, venlafaxine, dapoxetine, duloxetine, vilazodone, nefazodone, imipramine, femoxetine and clomipramine.

UCL 1283 and an SRI selected from citalopram, escitalopram, fluoxetine, sertraline, paroxetine, fluvoxamine, venlafaxine, dapoxetine, duloxetine, vilazodone, nefazodone, imipramine, femoxetine and clomipramine.

UCL 1390 and an SRI selected from citalopram, escitalopram, fluoxetine, sertraline, paroxetine, fluvoxamine, venlafaxine, dapoxetine, duloxetine, vilazodone, nefazodone, imipramine, femoxetine and clomipramine.

UCL 1409 and an SRI selected from citalopram, escitalopram, fluoxetine, sertraline, paroxetine, fluvoxamine, venlafaxine, dapoxetine, duloxetine, vilazodone, nefazodone, imipramine, femoxetine and clomipramine.

UCL 1860 and an SRI selected from citalopram, escitalopram, fluoxetine, sertraline, paroxetine, fluvoxamine, venlafaxine, dapoxetine, duloxetine, vilazodone, nefazodone, imipramine, femoxetine and clomipramine.

UCL 1972 and an SRI selected from citalopram, escitalopram, fluoxetine, sertraline, paroxetine, fluvoxamine, venlafaxine, dapoxetine, duloxetine, vilazodone, nefazodone, imipramine, femoxetine and clomipramine.

UCL 2065 and an SRI selected from citalopram, escitalopram, fluoxetine, sertraline, paroxetine, fluvoxamine, venlafaxine, dapoxetine, duloxetine, vilazodone, nefazodone, imipramine, femoxetine and clomipramine.

UCL 2138 and an SRI selected from citalopram, escitalopram, fluoxetine, sertraline, paroxetine, fluvoxamine, venlafaxine, dapoxetine, duloxetine, vilazodone, nefazodone, imipramine, femoxetine and clomipramine.

UCL 2173 and an SRI selected from citalopram, escitalopram, fluoxetine, sertraline, paroxetine, fluvoxamine, venlafaxine, dapoxetine, duloxetine, vilazodone, nefazodone, imipramine, femoxetine and clomipramine.

UCL 2283 and an SRI selected from citalopram, escitalopram, fluoxetine, sertraline, paroxetine, fluvoxamine, venlafaxine, dapoxetine, duloxetine, vilazodone, nefazodone, imipramine, femoxetine and clomipramine.

Verongamine and an SRI selected from citalopram, escitalopram, fluoxetine, sertraline, paroxetine, fluvoxamine, venlafaxine, dapoxetine, duloxetine, vilazodone, nefazodone, imipramine, femoxetine and clomipramine.

In a final embodiment, the present invention relates to a method for the identification of compounds useful for the treatment of depression, anxiety disorders and other affective disorders, such as generalized anxiety disorder, panic anxiety, obsessive compulsive disorder, acute stress disorder, post traumatic stress disorder and social anxiety disorder, eating disorders such as bulimia, anorexia and obesity, phobias, dysthymia, premenstrual syndrome, cognitive disorders, impulse control disorders, attention deficit hyperactivity disorder, drug abuse or any other disorder responsive to serotonin reuptake inhibitors, comprising, in any order:
(a) measuring the ability of test compounds to inhibit serotonin reuptake and selecting the compounds that have an IC₅₀ value below 50 nM;
(b) measuring the affinity of test compounds to the H₃ receptor and selecting the compounds.
   and thereafter measuring the efficacy of the selected compounds at the H₃ receptor and selecting the compounds which are antagonists, inverse agonists at the receptor.

Preferred H₃ ligands show affinity of below 0.5 μM, whereas other preferred ligands show affinity of below 0.1 μM and yet other preferred ligands show affinity of below 50 nM. Even more preferred are compounds with affinity below 10 nM.

Examples of assays for the selection/detection of H₃ antagonists, inverse agonists or partial agonists are for example the following:

Binding assay for the detection of compounds with affinity for H₃ receptors are described in Wulff B., Shastrup S., Rimvall K., European Journal of Pharmacology, vol. 453., pp 33-41 (2002).

Efficacy assay for the detection of antagonists, partial agonists or inverse agonists at the H₃ receptors are for example: Wulff B., Shastrup S., Rimvall K., European Journal of Pharmacology, vol. 453., pp 33-41 (2002).

The invention also covers compounds identified according to this method, but is not limited to these assay methods.

According to the invention, it has been found that co-administration of H₃ receptor antagonist or inverse agonist and a serotonin reuptake inhibitor produces a significant increase in the level of serotonin in terminal areas, as measured in microdialysis experiments, compared to the administration of the serotonin reuptake inhibitor alone.

According to the invention, animal studies have shown that H₃ receptor antagonist or inverse agonist may provide fast onset of therapeutic effect of serotonin reuptake inhibitors and potentiate the anxiolytic potential of serotonin reuptake inhibitors.

The use of a combination of H₃ receptor antagonist, inverse agonist or partial agonist and a serotonin reuptake inhibitor may greatly reduce the amount of serotonin reuptake inhibitor necessary to treat depression and other affective disorders and may thus reduce the side effects caused by the serotonin reuptake inhibitor. In particular, the combination of a reduced amount of SRI and a H₃ receptor antagonist, inverse agonist or partial agonist may reduce the risk of SRI-induced sexual dysfunction and sleep disturbances.

Co-administration of a H₃ receptor antagonist, inverse agonist or partial agonist and a serotonin reuptake inhibitor may also be useful for the treatment of refractory depression, i.e. depression, which cannot be treated appropriately by administration of a serotonin reuptake inhibitor alone. Typically, H₃ receptor antagonist, inverse agonist or partial agonist may be used as add-on therapy for the augmentation of the response to SRIs in patients where at least 40-60% reduction in symptoms has not been achieved during the first 6 weeks of treatment with an SRI.

Compounds which are both serotonin reuptake inhibitors and H₃ receptor antagonists, inverse agonists or partial agonists may have the same pharmacological advantages as the combination of a serotonin reuptake inhibitor and a H₃ receptor antagonists, inverse agonists or partial agonists, with respect to reduction of side effects, fast onset and in the treatment of treatment resistant patients.

Many antidepressants with serotonin reuptake inhibiting effect have been described in the literature. Any pharmacologically active compound, which primarily or partly exerts its therapeutic effect via inhibition of serotonin reuptake in the CNS, may benefit from augmentation with a H₃ receptor antagonist, inverse agonist or partial agonist.

The following list contains a number of serotonin reuptake inhibitors, which may benefit from augmentation with a H₃ receptor antagonist, inverse agonist or partial agonist: citalopram, escitalopram, fluoxetine, R-fluoxetine, sertraline, paroxetine, fluvoxamine, venlafaxine, desmethylvenlafaxine, duloxetine, dapoxetine, vilazodone, nefazodone, imipramine, imipramine N-oxide, desipramine, pirandamine, dazepinil, nefopam, befuraline, fezolamine, femoxetine, clomipramine, cianoimipramine, litoxetine, cericlamine, seproxetine, WY 27587, WY 27866, imeidine, ifoxetine, indeloxazine, tiflucarbine, viqualine, milnacipran, bazinaprine, YM 922, S 33005, F 98214-TA, FI 4503, A 80426, EMD 86006, NS 2389, S33005, OPC 14523, alaproclate, cyanodothepine, trimipramine, quinupramine, dothiepin, amoxapine, nitroxazepine, McN 5652, McN 5707, VN 2222, L 792339, roxindole, YM 35992, O1 77, Org 6582, Org 6997, Org 6906, amitriptyline, amitriptyline N-oxide, nortriptyline, CL 255.663, pirlindole, indatraline, LY 280253, LY 285974, LY 113.821, LY 214.281, CGP 6085 A, RU 25.591, napamezole, diclofensine, trazodone, BMY 42.569, NS 2389, sercloremine, nitroquipazine, ademethionine, sibutramine, desmethylsubitramine, didesmethylsubitramine, clovoxamine vilazodone. The compounds mentioned above may be used in the form of the base or a pharmaceutically acceptable acid addition salt thereof Each of the serotonin reuptake inhibitors specified above is intended to be an individual embodiment. Accordingly, each of them and the use thereof may be claimed individually.

Compounds such as citalopram, escitalopram, fluoxetine, R-fluoxetine, sertraline, paroxetine, fluvoxamine, venlafaxine, desmethylvenlafaxine, duloxetine, dapoxetine, vilazodone, nefazodone, imipramine, imipramine N-oxide, desipramine, pirandamine, dazepinil, nefopamn, befuraline, fezolamine, femoxetine, clomipramine, cianoimipramine, litoxetine, cericlamine, seproxetine, imeldine, ifoxetine, indeloxazine, tiflucarbine, viqualine, milnacipran, bazinaprine, alaproclate, cyanodothepine, trimipramine, quinupramine, dothiepin, amoxapine, nitroxazepine, roxindole, amitriptyline, amitriptyline N-oxide, nortriptyline, pirlindole, indatraline, napamezole, diclofensine, trazodone, sercloremine, nitroquipazine, ademethionine, sibutramine, desmethylsubitramine, didesmethylsubitramine, clovoxamine vilazodone,

N-[(1-[(6-Fluoro-2-naphthalenyl)methyl]-4-piperidinyl]amino]carbonyl]-3-pyridine carboxamide (WY 27587),

[trans-6-(2-chlorophenyl)-1,2,3,5,6,10b-hexahydropyrrolo-(2,1-a)isoquinoline](McN 5707),

(dl-4-exo-amino-8-chloro-benzo-(b)-bicyclo[3.3.1]nona-2-6 alpha(10 alpha)-diene hydrochloride)(Org 6997),

(dl)-(5 alpha,8 alpha,9 alpha)-5,8,9,10-Tetrahydro-5,9-methanobenzocycloocten-8-amine hydrochloride (Org 6906),

-[2-[4-(6-fluoro-1H-indol-3-yl)-3,6-dihydro-1(2H)-pyridinyl]ethyl]-3-isopropyl-6-(methylsulphonyl)-3,4-dihydro-1H-2,1,3-benzothiadiazine-2,2-dioxide (LY393558),

[4-(5,6-dimethyl-2-benzofuranyl)-piperidine](CGP 6085),

dimethyl-[5-(4-nitro-phenoxy)-6,7,8,9-tetrahydro-5H-benzocyclohepten-7-yl]-amine (RU 25.591), 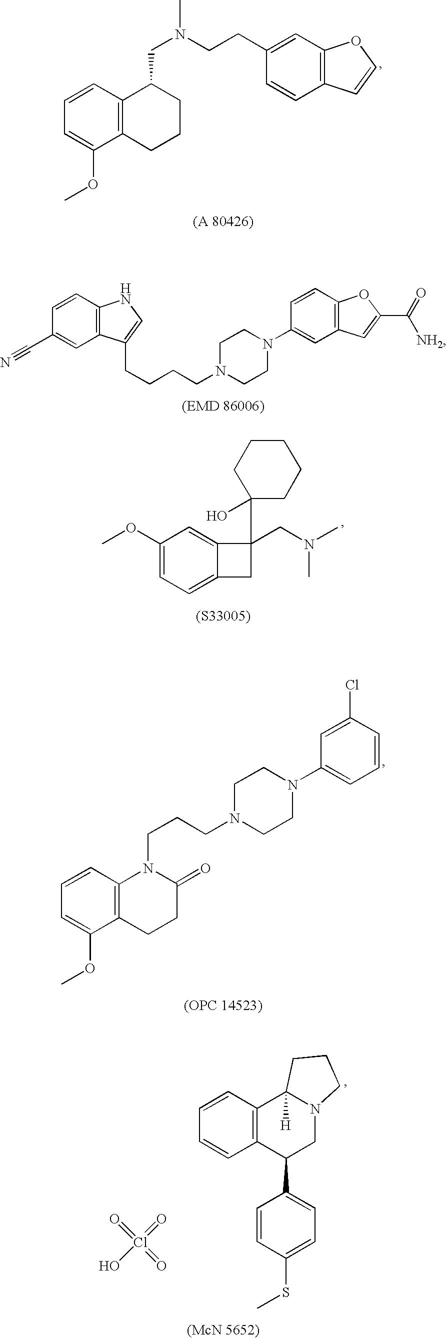 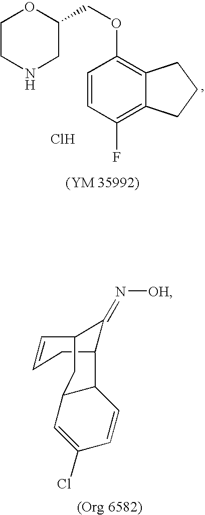
are preferred. The compounds mentioned above may be used in the form of the base or a pharmaceutically acceptable acid addition salt thereof. Each of the serotonin reuptake inhibitors specified above is intended to be an individual embodiment. Accordingly, each of them and the use thereof may be claimed individually.

Other therapeutic compounds, which may benefit from augmentation with a H₃ receptor antagonists, inverse agonist or partial agonists, include compounds, which cause an elevation in the extracellular level of 5-HT in the synaptic cleft, although they are not serotonin reuptake inhibitors. One such compound is tianeptine.

Accordingly, other compounds than SRIs which cause an elevation in the extracellular level of serotonin, may be used instead of SRIs in every aspect of the invention as described herein.

The above list of serotonin reuptake inhibitors and other compounds, which cause an increase in the extracellular level of serotonin, may not be construed as limiting.

SRIs, which are particularly preferred according to the present invention, include citalopram, escitalopram, fluoxetine, sertraline, paroxetine, fluvoxarnine, venlafaxine, dapoxetine, duloxetine, vilazodone, nefazodone, imipramine, femoxetine and clomipramine.

The term selective serotonin reuptake inhibitor (SSRI) means an inhibitor of the monoamine transporters, which has stronger inhibitory effect at the serotonin transporter than the dopamine and the noradrenaline transporters. Particularly preferred SSRIs according to the invention are citalopram, escitalopram, fluoxetine, fluvoxamine, sertraline, duloxetine, vilnazodone and paroxetine.

In particular individual embodiments, citalopram or escitalopram is used.

The following list contains a number of H₃ antagonists, partial agonists or inverse agonists, which may be used according to the invention. Each of the H₃ antagonists, partial agonists or inverse agonists specified above is intended to be an individual embodiment. Accordingly, each of them may be claimed individually.

Whenever mentioned, each of the terms “H₃ antagonist, partial agonist or inverse agonist”, “H₃ receptor antagonist, partial agonist or inverse agonist”, “H₃ ligand”, and “H₃ receptor ligand” means H₃ receptor antagonist, partial H₃ receptor agonist and inverse H₃ receptor agonist. Each of which is intended to be an individual embodiment. Accordingly, each of these embodiments and the use thereof may be claimed individually.

A particular embodiment relates to a H₃ receptor antagonist and the use thereof.

Pharmaceutical Compositions

Each of the active ingredients according to the invention may be administered alone or together or in combination with pharmaceutically acceptable carriers or excipients, in either single or multiple doses. The pharmaceutical compositions according to the invention may be formulated with pharmaceutically acceptable carriers or diluents as well as any other known adjuvants and excipients in accordance with conventional techniques such as those disclosed in Remington: The Science and Practice of Pharmacy, 19 Edition, Gennaro, Ed., Mack Publishing Co., Easton, Pa., 1995.

The pharmaceutical compositions may be specifically formulated for administration by any suitable route such as the oral, rectal, nasal, pulmonary, topical (including buccal and sublingual), transdermal, intracisternal, intraperitoneal, vaginal and parenteral (including subcutaneous, intramuscular, intrathecal, intravenous and intradermal) route, the oral route being preferred. It will be appreciated that the preferred route will depend on the general condition and age of the subject to be treated, the nature of the condition to be treated and the specific active ingredient or active ingredients chosen.

Pharmaceutical compositions for oral administration include solid dosage forms such as capsules, tablets, dragees, pills, lozenges, powders and granules. Where appropriate, they may be prepared with coatings such as enteric coatings or they may be formulated so as to provide controlled release of one or more active ingredient such as sustained or prolonged release according to methods well known in the art.

Liquid dosage forms for oral administration include solutions, emulsions, suspensions, syrups and elixirs.

Pharmaceutical compositions for parenteral administration include sterile aqueous and nonaqueous injectable solutions, dispersions, suspensions or emulsions as well as sterile powders to be reconstituted in sterile injectable solutions or dispersions prior to use. Depot injectable formulations are also contemplated as being within the scope of the present invention.

Other suitable administration forms include suppositories, sprays, ointments, cremes, gels, inhalants, dermal patches, implants etc.

The pharmaceutical compositions of this invention or those which are manufactured in accordance with this invention may be administered by any suitable route, for example orally in the form of tablets, capsules, powders, syrups, etc., or parenterally in the form of solutions for injection. For preparing such compositions, methods well known in the art may be used, and any pharmaceutically acceptable carriers, diluents, excipients or other additives normally used in the art may be used.

A typical oral dosage of each of the active ingredients is in the range of from about 0.001 to about 100 mg/kg body weight per day, preferably from about 0.01 to about 50 mg/kg body weight per day, and more preferred from about 0.05 to about 10 mg/kg body weight per day administered in one or more dosages such as 1 to 3 dosages. The exact dosage will depend upon the frequency and mode of administration, the sex, age, weight and general condition of the subject treated, the nature and severity of the condition treated and any concomitant diseases to be treated and other factors evident to those skilled in the art.

For parenteral routes such as intravenous, intrathecal, intramuscular and similar administration, typically doses are in the order of about half the dose employed for oral administration.

The compounds of this invention are generally utilized as the free substance or as a pharmaceutically acceptable salt thereof. One example is a base addition salt of a compound having the utility of a free acid. When an active ingredient contains a free acid such salts are prepared in a conventional manner by treating a solution or suspension of a free acid of the active ingredient with a chemical equivalent of a pharmaceutically acceptable base.

For parenteral administration, solutions of one or more active ingredient in sterile aqueous solution, aqueous propylene glycol, aqueous vitamin E or sesame or peanut oil may be employed. Such aqueous solutions should be suitably buffered if necessary and the liquid diluent first rendered isotonic with sufficient saline or glucose. The aqueous solutions are particularly suitable for intravenous, intramuscular, subcutaneous and intraperitoneal administration. The sterile aqueous media employed are all readily available by standard techniques known to those skilled in the art.

Solutions for injections may be prepared by dissolving one or more active ingredients and possible additives in a part of the solvent for injection, preferably sterile water, adjusting the solution to a desired volume, sterilising the solution and filling it in suitable ampules or vials. Any suitable additive conventionally used in the art may be added, such as tonicity agents, preservatives, antioxidants, etc.

Suitable pharmaceutical carriers include inert solid diluents or fillers, sterile aqueous solution and various organic solvents.

Examples of solid carriers are lactose, terra alba, sucrose, cyclodextrin, talc, agar, pectin, acacia, stearic acid and lower alkyl ethers of cellulose corn starch, potato starch, talcum, magnesium stearate, gelatine, lactose, gums, and the like.

Any other adjuvants or additives usually used for such purposes such as colourings, flavourings, preservatives etc. may be used provided that they are compatible with the active ingredient or ingredients used.

Examples of liquid carriers are syrup, peanut oil, olive oil, phospho lipids, fatty acids, fatty acid amines, polyoxyethylene and water. Similarly, the carrier or diluent may include any sustained release material known in the art, such as glyceryl monostearate or glyceryl distearate, alone or mixed with a wax.

The pharmaceutical compositions formed by combining one or more active ingredients of the invention with the pharmaceutical acceptable carriers are then readily administered in a variety of dosage forms suitable for the disclosed routes of administration. The formulations may conveniently be presented in unit dosage form by methods known in the art of pharmacy.

The active ingredients of the invention may be formulated in similar or dissimilar pharmaceutical compositions and unit forms thereof.

If a solid carrier is used for oral administration, the preparation may be tablette, placed in a hard gelatine capsule in powder or pellet form or it may be in the form of a troche or lozenge.

The amount of solid carrier will vary widely but will usually be from about 25 mg to about 1 g.

If a liquid carrier is used, the preparation may be in the form of a syrup, emulsion, soft gelatine capsule or sterile injectable liquid such as an aqueous or non-aqueous liquid suspension or solution.

If desired, the pharmaceutical composition of the invention may comprise one or more active ingredients in combination with frrther pharmacologically active substances such as those described in the foregoing.

### MATERIALS AND METHODS

Animals

Male albino rats of a Wistar-derived strain (285-320 g; Harlan, Zeist, The Netherlands) were used for the experiments. Upon surgery, rats were housed individually in plastic cages (35×35×40 cm), and had free access to food and water. Animals were kept on a 12 h light schedule (light on 7:00 a.m.). The experiments are concordant with the declarations of Helsinki and were approved by the animal care committee of the faculty of mathematics and natural science of the University of Groningen.

Drugs

The following drugs were used: Citalopram hydrobromide, Thioperamide (Sigmna, St Louis, USA). Ciproxifan (synthesized at Lundbeck A/S).

Surgery

Microdialysis of brain serotonin levels was performed using home made I-shaped probes, made of polyacrylonitrile/sodium methyl sulfonate copolymer dialysis fiber (i.d. 220 μm, o.d. 300 μm, AN 69, Hospal, Italy). Preceding surgery rats were anaesthetised using isoflurane (O₂/N₂O; 300/300 ml/min). Lidocaine-HCl, 10 % (m/v) was used for local anaesthesia. Rats were placed in a stereotaxic frame (Kopf, USA), and probes were inserted into Ventral Hippcampus (V. Hippo, L +4.8 mm, IA: +3.7 30 mm, V: −8.0 mm) and median prefrontal cortex (PFC, L −0.9 mm; AP: +3.5 mm relative to bregma; V: −6.0 mm (Paxinos and Watson, 1982). After insertion, probes were secured with dental cement.

Microdialysis Experiments

Rats were allowed to recover for at least 24 h. Probes were perfused with artificial cerebrospinal fluid containing 147 mM NaCl, 3.0 mM KCl, 1.2 mM CaCl₂, and 1.2 mM MgCl₂, at a flow-rate of 1.5 μl/min (Harvard apparatus, South Natick, Mass., USA). 15 minute microdialysis samples were collected in HPLC vials containing 7.5 μl 0.02 M acetic acid for serotonin analysis.

Serotonin Analysis:

Twenty-μl microdialysate samples were injected via an autoinjector (CMA/200 refrigerated microsampler, CMA, Sweden) onto a 100×2.0 mm C18 Hypersil 3 μm column (Bester, Amstelveen, the Netherlands) and separated with a mobile phase consisting of 5 g/L di-ammoniumsulfate, 500 mg/L EDTA, 50 mg/L heptane sulphonic acid, 4% methanol v/v, and 30 μI/L of triethylamine, pH 4.65 at a flow of 0.4 ml/min (Shimadzu LC-10 AD). 5-HT was detected amperometrically at a glassy carbon electrode at 500 mV vs Ag/AgCl (Antec Leyden, Leiden, The Netherlands). The detection limit was 0.5 fmol 5-HT per 20 μl sample (signal to noise ratio 3).

Data Presentation and Statistics

Four consecutive microdialysis samples with less then 20% variation were taken as control and set at 100%. Data are presented as percentages of control level (mean±S.E.M.) in time. Statistical analysis was performed using Sigmastat for Windows (SPSS, Jandel Corporation). Treatments were compared versus controls using two way analysis of variance (ANOVA) for repeated measurements, followed by Student Newman Keuls test. Level of significance level was set at p<0.05.

Results

Co-Administration of Citalopram with Thioperamide

Administration of 5 mg/kg s.c. thioperamide did not induce any effects on serotonin levels in ventral hippocampus (X¹₁₀=10, P =0.44 n.s.).

Co-administration of citalopram 10 μmol/kg with thioperamide (5 mg/kg s.c.) induced an augmented effect on 5-HT levels when compared to citalopram administration alone (Treatment vs. Time F(10,145)=6.48, P<0.0001). Post-hoc analysis revealed significant differences in time at 75, 90, 105 and 150 min after injection.

Co-Administration of Citalopram With Ciproxifan

Administration of 15 mg/kg s.c. ciproxifan did not induce any effects on serotonin levels in ventral hippocampus (X¹₁₀=8.84, P=0.54 n.s.).

Co-administration of citalopram 10 μmol/kg with ciproxifan (15 mg/kg s.c.) induced an augmented effect on 5-HT levels when compared to citalopram administration alone (Treatment vs. Time F(10,147)=8.90, P<0.0001). Post-hoc analysis revealed significant differences in time at 60, 75, 90, 105, 120 and 135 min after injection.

1. A pharmaceutical composition comprising one compound, which is a serotonin reuptake inhibitor, and another compound, which is a H₃ receptor antagonist, inverse agonist or partial agonist.

2. A pharmaceutical composition comprising a compound that is both a H₃ receptor antagonist, inverse agonist or partial agonist and a serotonin reuptake inhibitor.

3. A method of augmenting and/or providing faster onset of the therapeutic effect of a serotonin reuptake inhibitor, comprising administering to a patient in need thereof a therapeutically effective amount of a H₃ receptor antagonist, inverse agonist or partial agonist.

4. A method of treating depression or an affective disorder, comprising administering a therapeutically effective amount of a H₃ receptor antagonist, inverse agonist or partial agonist to a patient being treated with a serotonin reuptake inhibitor and in need thereof.

5. A method of treating depression or an affective disorder, comprising administering to a patient in need thereof a therapeutically effective amount of a pharmaceutical composition according to claim 1.

6. The method of claim 5, wherein the compound is a selective serotonin reuptake inhibitor.

7. The method of claim 5, wherein the compound is selective for the H₃ receptor.

8. The method of claim 5, wherein the compound is an antagonist or an inverse agonist at the H₃ receptor.

9. The method of claim 8, wherein the compound is a H₃ receptor antagonist.

10. The method of claim 6, wherein the serotonin reuptake inhibitor is selected from citalopram, escitalopram, fluoxetine, sertraline, paroxetine, fluvoxamine, venlafaxine, dapoxetine, duloxetine, vilazodone, nefazodone, imipramine, femoxetine, and clomipramine.

11. The method of claim 5, wherein the H3 receptor ligand is selected from Thioperamide, Ciproxifan, Iodophenpropit, GR 175737, Iodoproxyfan, Proxifan, Perceptin, JB 98064, VUF 9153, A 304121, ABT923, ABT 834, A 923, A 320436, A 331440, A 349413, A 349821, A 417022, A 423579, A 424835, A 431404, AQ 0145, FUB 181, FUB 360, FUB 407, FUB 637, FUB 836, GR 168320, GSK 189254A, GSK 207040A, GT 2016, GT 2104, GT 2209, GT 2212, GT 2227, GT 2232, GT 2390, GT 2349, GT 2355, GT 2394, Imoproxifan, Impentamine, JNJ 5207852, NNC 0038 0000 1049, NNC 0038 0000 1202, SCH 50971, SCH 79687, UCL 1199, UCL 1283, UCL 1390, UCL 1409, UCL 1860, UCL 1972, UCL 2065, UCL 2138, UCL 2173, UCL 2283, Verongamine, VUF 4163, VUF 5000, and VUF 5182.

12. The pharmaceutical composition according to claim 1, further comprising a pharmaceutically acceptable carrier or diluent diluents.

13. The pharmaceutical composition according to claim 1, wherein the serotonin reuptake inhibitor used is a selective serotonin reuptake inhibitor.

14. The pharmaceutical composition according to claim 1, wherein the H₃ antagonist, inverse agonist or partial agonist is selective for the H₃ receptor.

15. The pharmaceutical composition according to claim 1, wherein the H₃ ligand is a H₃ receptor antagonist.

16. The pharmaceutical composition according to claim 1, wherein the serotonin uptake inhibitor is selected from citalopram, escitalopram, fluoxetine, sertraline, paroxetine, fluvoxamine, venlafaxine, dapoxetine, duloxetine, vilazodone, nefazodone, imipramine, femoxetine and clomipramine.

17. A pharmaceutical composition according to claim 1, wherein the H₃ ligand is selected from from Thioperamide, Ciproxifan, Iodophenpropit, GR 175737, Iodoproxyfan, Proxifan, Perceptin, JB 98064, VUF 9153, A 304121, ABT923, ABT 834, A 923, A 320436, A 331440, A 349413, A 349821, A 417022, A 423579, A 424835, A 431404, AQ 0145, FUB 181, FUB 360, FUB 407, FUB 637, FUB 836, GR 168320, GSK 189254A, GSK 207040A, GT 2016, GT 2104, GT 2209, GT 2212, GT 2227, GT 2232, GT 2390, GT 2349, GT 2355, GT 2394, Imoproxifan, Impentamine, JNJ 5207852, NNC 0038 0000 1049, NNC 0038 0000 1202, SCH 50971, SCH 79687, UCL 1199, UCL 1283, UCL 1390, UCL 1409, UCL 1860, UCL 1972, UCL 2065, UCL 2138, UCL 2173, UCL 2283, Verongamine, VUF 4163, VUF 5000, and VUF 5182.

18. The method of claim 5, wherein the active ingredients are administered by simultaneous administration.

19. The method of claim 5, wherein the active ingredients are administered in the same unit dosage form.

20. The method of claim 5, wherein the active ingredients are administered by sequential administration.

21. The method of claim 5, wherein the active ingredients are administered in discrete dosage forms.

22. A method for identifying compounds useful for the treatment of depression or an affective disorder, comprising, in any order:
(a) measuring the ability of test compounds to inhibit serotonin reuptake and selecting the compounds that have an IC₅₀ value below 50 nM;
(b) measuring the affinity of test compounds to the H₃ receptor and selecting the compounds that have an affinity,
and thereafter measuring the efficacy of the selected compounds at the H₃ receptor and selecting the compounds which are antagonists, inverse agonists or partial agonists at the receptor.

23. The method according to claim 22 wherein the compound has an affinity in step (b) of less than 50 nM.

24. The method according to claim 23, wherein the compound has an affinity in step (b) of less than 10 nM.

25. A compound that inhibits serotonin reuptake and has an IC₅₀value below 50 nM, and has an affinity to the H₃ receptor.

26. A method of treating depression or an affective disorder, comprising administering to a patient in need thereof a therapeutically effective amount of a pharmaceutical composition according to claim 2.

27. The method of claim 26, wherein the serotonin reuptake inhibitor is a selective serotonin reuptake inhibitor.

28. The method of claim 26, wherein the H₃ receptor antagonist, inverse agonist or partial agonist is selective for the H₃ receptor.

29. The method of claim 26, wherein the H₃ receptor antagonist, inverse agonist or partial agonist is an antagonist or an inverse agonist at the H₃ receptor.

30. The method of claim 29, wherein the H₃ receptor antagonist, inverse agonist or partial agonist is a H₃ receptor antagonist.

31. The pharmaceutical composition according to claim 2, further comprising a pharmaceutically acceptable carrier or diluent.

32. The pharmaceutical composition according to claim 2, wherein the H₃ ligand is a H₃ receptor antagonist.

33. The method of claim 4, wherein the affective disorder is selected from anxiety disorders, generalized anxiety disorder, panic anxiety, obsessive compulsive disorder, acute stress disorder, post traumatic stress disorder, social anxiety disorder, eating disorders, phobias, dysthymia, premenstrual syndrome, cognitive disorders, impulse control disorders, attention deficit hyperactivity disorder, drug abuse or any other disorder responsive to a serotonin reuptake inhibitor.

34. The method of claim 33, wherein the eating disorder is selected from, bulimia, anorexia, and obesity.

35. The method of claim 5, wherein the affective disorder is selected from anxiety disorders, generalized anxiety disorder, panic anxiety, obsessive compulsive disorder, acute stress disorder, post traumatic stress disorder, social anxiety disorder, eating disorders, phobias, dysthymia, premenstrual syndrome, cognitive disorders, impulse control disorders, attention deficit hyperactivity disorder, drug abuse or any other disorder responsive to a serotonin reuptake inhibitor.

36. The method of claim 35, wherein the eating disorder is selected from, bulimia, anorexia, and obesity.

37. The method of claim 22, wherein the affective disorder is selected from anxiety disorders, generalized anxiety disorder, panic anxiety, obsessive compulsive disorder, acute stress disorder, post traumatic stress disorder, social anxiety disorder, eating disorders, phobias, dysthymia, premenstrual syndrome, cognitive disorders, impulse control disorders, attention deficit hyperactivity disorder, drug abuse or any other disorder responsive to a serotonin reuptake inhibitor.

38. The method of claim 37, wherein the eating disorder is selected from bulimia, anorexia, and obesity.

39. The method of claim 26, wherein the affective disorder is selected from anxiety disorders, generalized anxiety disorder, panic anxiety, obsessive compulsive disorder, acute stress disorder, post traumatic stress disorder, social anxiety disorder, eating disorders, phobias, dysthymia, premenstrual syndrome, cognitive disorders, impulse control disorders, attention deficit hyperactivity disorder, drug abuse or any other disorder responsive to a serotonin reuptake inhibitor.

40. The method of claim 39, wherein the eating disorder is selected from, bulimia, anorexia, and obesity.

41. The compound of claim 25, wherein the compound has an affinity to the H₃ receptor of less than 50 nM.

42. The compound of claim 41, wherein the compound has an affinity to the H₃ receptor of less than 10 nM.
